(19) United States
(12) Patent Application Publication
(10) Pub. No.: US 2013/0302265 A1
(43) Pub. Date: Nov. 14, 2013

(54) TOPICAL COMPOSITION AND METHOD FOR SKIN LIGHTENING

(71) Applicant: ACCESS BUSINESS GROUP INTERNATIONAL LLC, Ada, MI (US)

(72) Inventors: Jatinder Rana, Grand Rapids, MI (US); Ganesh Diwakar, Grand Rapids, MI (US); Han Qiang, Shanghai (CN); Tingzhao Li, Shanghai (CN); Jeffrey Scholten, Grand Rapids, MI (US)

(73) Assignee: Access Business Group International LLC, Ada, MI (US)

(21) Appl. No.: 13/887,729

(22) Filed: May 6, 2013

Related U.S. Application Data
(60) Provisional application No. 61/644,684, filed on May 9, 2012.

(51) Int. Cl. A61K 8/97; A61Q 19/02

(52) U.S. Cl. CPC A61K 8/97; A61Q 19/02
USPC 424/62

(57) ABSTRACT

A topical cosmetic composition for skin whitening or skin lightening is described. The composition includes an aqueous extract of Caesalpinia spinosa, also known as Tara, in an amount effective to reduce melanin production. The composition may also include an extract of Centella asiatica, also known as Gotu Kola. A method for using the compositions is also described.

### CROSS REFERENCE TO RELATED APPLICATION

This application claims the benefit of U.S. Provisional Application No. 61/644,684, filed May 9, 2012.

### BACKGROUND OF THE INVENTION

The present invention relates to topical skin lightening, or skin whitening compositions.

Human skin coloration is determined by the amount and location of melanin in the surface of the skin. Melanin is synthesized by the oxidation of the amino acid tyrosine to L-3,4-dihydroxyphenylalanine (L-DOPA) in cells commonly known as melanocytes that are present at the dermis-epidermis junction. The oxidation process is catalyzed by an enzyme known as tyrosinase. The series of cellular processes carried out by melanocytes is commonly known as melanogenesis.

Skin coloration is regulated by the amount and type of melanin synthesized by the melanocytes. Environmental factors can also affect skin color. A healthy amount of melanin in the skin is effective at absorbing ultraviolet (“UV”) rays. Increased exposure of the skin to UV rays generally increases the amount and rate of melanin production and can result in darker skin, or a “tan.” Pigmentation disorders such as hyperpigmentation or hypopigmentation, either localized or general, can result from a number of factors including hormone levels in the body, diet, genetic disorders, and medications. Common pigmentation disorders include melisma, liver spots and vitiligo.

Various preparations have been formulated to address pigmentation disorders and have, for example, been intended for use in the treatment of hyperpigmentation and/or hypopigmentation. Such treatments are typically referred to by as “skin lightening,” “skin whitening” or “skin brightening”. There are several uses for skin whiteners. For example, to lighten age spots (liver spots or senile lentigo), or to prevent the darkening of Caucasian and Asian skins (that is, to maintain a light/fair complexion). Some of these preparations have included tyrosinase inhibitors such as hydroquinone, vitamin C, kojic acid, arbutin, glutathione, cysteine, lactic acid, ferulic acid, nicotinamide and plant extracts such as bearberry and mulberry extract, etc. However, some of these compounds, such as hydroquinone and kojic acid, have side effects including skin irritation, acute dermatitis and cytotoxicity of skin cells.

In general, there remains a need for additional skin lightening agents, especially ones of high efficacy. Most especially, there remains a need for skin lightening agents that are efficacious and incorporate natural materials into compositions to address some of these issues. In particular, there remains a need for skin lightening compositions based on synergistic combinations of materials for skin lightening.

### SUMMARY OF THE INVENTION

A topical composition provides a reduction in melanin production. The composition can include an effective amount of an extract derived from the plant genus Caesalpinia, for example, Caesalpinia spinosa, which is also known as Tara. The composition can whiten or lighten skin.

In one embodiment, the composition includes an extract obtained from the fruit pod of the Caesalpinia plant.

Another aspect of the invention includes the composition in a cosmetically acceptable vehicle such as water, glycerin, waxes, an alcohol, vegetable oil, mineral oil, silicone, fatty esters, fatty alcohols, glycols, polyglycols or combinations thereof.

In another embodiment, the composition includes an extract of Centella asiatica, which is also known as Gotu Kola.

In a further embodiment, the extract of Caesalpinia and Centella asiatica are present in the composition in a 1:1 concentration ratio.

The invention also relates to a method for whitening or lightening skin that includes applying to the skin compositions described according to the invention in an amount and for a time sufficient to whiten or lighten skin.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 shows an exemplary process for producing an extract from a Caesalpinia, or Tara, plant.

FIG. 2 is graphical representation of the dose-dependent reduction in melanin production with Tara extract.

FIG. 3 is a graphical representation of the synergistic reduction in melanin production achieved by the combination of Tara extract and Centella asiatica, or Gotu Kola, extract.

FIG. 4 is a graphical representation of the results from a cell viability assay.

### DESCRIPTION OF THE CURRENT EMBODIMENT

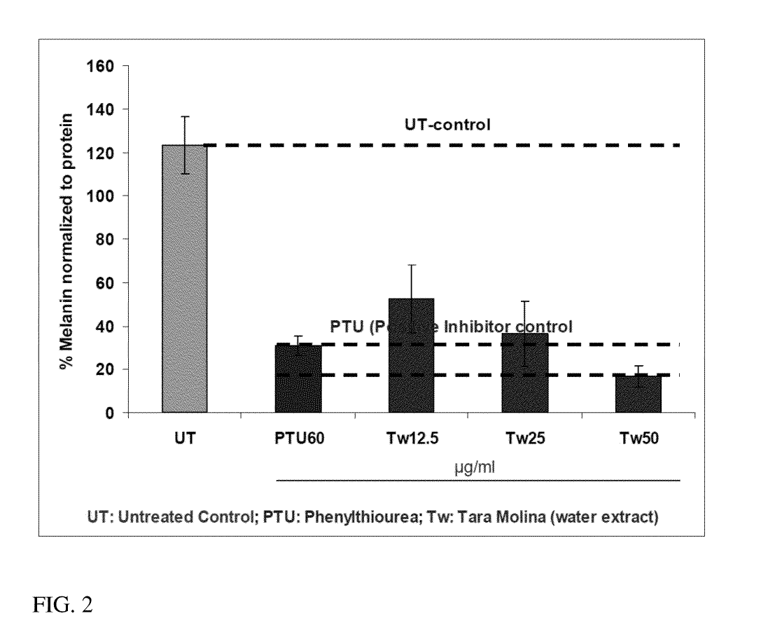

The current embodiments are based on the surprising discovery that extracts of genus Caesalpinia inhibit melanin production. The particular composition investigated included an extract of a plant from the genus, Caesalpinia, specifically Caesalpinia spinosa, which also is referred to as “Tara”, or alternatively as Molina, Kuntze or Spiny Holdback. The compositions demonstrated a dose-dependent negative effect on the production of melanin. In particular, as shown in FIG. 2, when administered to melanocytes, the composition of Tara extract provided an increasing reduction of melanin production with increasing doses of Tara extract. Based on the discoveries herein, a current embodiment is directed to a topical composition that includes an extract of a plant of the genus Caesalpinia, for example, Caesalpinia spinosa, “Tara” in amount sufficient to reduce melanin production in a subject, and optionally in a subject in need thereof.

As noted above, the composition can include extracts from Caesalpinia spinosa plants, however, it is contemplated that other plants of the Caesalpinia genus, such as Caesalpinia cacalaco, Caesalpinia californica, Caesalpinia gilliesii, Caesalpinia mexicana, Caesalpinia platyloba, and Caesalpinia pulcherrima also can provide a reduction in melanin production in a subject.

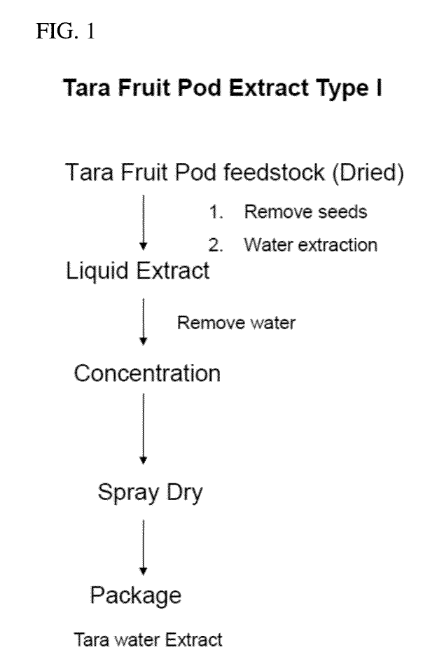

Generally, any part of the Caesalpinia plant may be used to produce the extract including, but not limited to, the root, stem, leaf, flower, fruit and fruit pod. One or more parts of the plant may be extracted to yield the Caesalpinia extract. The Tara extract used herein may be commercially obtained from various resources, including but not limited to, Yunnan Yimen Mingxin Tara Biotechnology Development Co., Ltd. of Yunan, China. In addition, any suitable Tara extracts can be obtained by using any conventional extraction techniques. As one example, the Tara extract may be obtained according to the process outlined in FIG. 1. In this process, Tara fruit pods are collected, dried and removed of seeds. Thereafter, the material is extracted with water. The water is removed in a concentration step, for example by pressing, and the material may be spray dried and packaged. The Tara extracts may be incorporated into the composition in an amount from about 0.01% to about 2%, preferably between about 0.05% and about 0.5%.

The amount of extract present in a skin whitening composition will depend upon several factors, including the desired level of melanin inhibition, the melanin inhibiting level in a particular extract or composition, and other factors. Preferably, the extract may include between about 0.01% and about 20% (wt/wt) of the total composition. More preferably, the extract may include between about 0.050% and 10% (wt/wt) of the total composition. Additional extracts or ingredients for in the topical composition of the present invention are described in U.S. Pat. No. 5,747,006 to Dornoff et al., U.S. Pat. Nos. 5,980,904, 6,994,874, 7,060,304, 7,247,321, and 7,364,759 to Leverett et al., the disclosures of which are hereby incorporated by reference in their entirety.

Another aspect of the invention includes combining the Tara extract with a Centella material, for example from Centella asiatica, which is also known as Gotu Kola, Brahmi, Indian Pennywort, March Penny, Spadeleaf, Tiger's Herb and Pennywort. Surprisingly and unexpectedly, it was found that the combination provided greater than expected melanin inhibition and thus an increase in skin whitening or lightening.

Centella asiatica is a perennial plant in the parsley family and is native to India, Japan, China, South Africa, Sri Lanka, and the South Pacific. Any part of the Centella plant may be used to produce the material used in the compositions described herein including, but not limited to, the root, stem, leaf, flower, fruit and extracts of these parts. One or more parts of the plant may be used to produce the Gotu Kola material or may extracted to yield the Gotu Kola material. The Gotu Kola material used herein was obtained from Gotu kola (Centella asiatica) Wholesale Suppliers, Gotu kola Traders, gotu kola Distributors (Tamil Nadu, INDIA). In addition, any suitable Gotu Kola extracts can be obtained by using any conventional extraction techniques.

There are a variety of extraction methods that may be used to produce a Gotu Kola extract suitable for the composition described herein. These methods include, but are not limited to, the extraction methods disclosed in U.S. Pat. No. 7,897,184 to Rana et al., which is hereby incorporated by reference in its entirety and partially reproduced below with respect to some extraction methods. While extraction solvents described specifically mention ethanol, it should be understood that other alcohols such as, but not limited to, isopropyl alcohol, ethyl methyl alcohol may be used in addition to or as an alternative to ethanol. Exemplary alcoholic solvents include, but are not limited to, C1-C4 alcohols, such as methanol, ethanol, propanol, isopropanol, and butanol; hydro-alcohols or mixtures of alcohol and water, including hydroethanol; polyhydric alcohols such as proplylene glyclol and butylene glycol; and fatty alcohols. Any of these alcoholic solvents may be used.

In one example, an extract useful in the unique compositions of the present invention might be obtained using an organic solvent extraction technique. In another example, solvent sequential fractionation may be used to obtain an extract useful in the unique compositions of the present invention. Total hydro-ethanolic extraction techniques might also be used to obtain an extract useful in the unique compositions of the present invention. Generally, this is referred to as a lump-sum extraction. The extract generated in the process will contain a broad variety of phytochemicals present in the extracted material including fat and water soluble. Following collection of the extract solution, the solvent will be evaporated, resulting in the extract.

Total ethanol extraction may also be used in the present invention. This technique uses ethanol as the solvent. This extraction technique generates an extract that may include fat soluble and/or lipophilic compounds in addition to water soluble compounds.

Another example of an extraction technique that might be used to obtain an extract useful in the present invention is supercritical fluid carbon dioxide extraction (SFE). In this extraction procedure the material to be extracted is not exposed to any organic solvents. Rather, the extraction solvent is carbon dioxide, with our without a modifier, in super-critical conditions (>31.3° C. and >73.8 bar). Those of skill in the art will appreciate that temperature and pressure conditions can be varied to obtain the best yield of extract. This technique generates an extract of fat soluble and/or lipophilic compounds, similar to the total hexane and ethyl acetate extraction technique described above.

The compositions described herein may be formulated to include a cosmetically acceptable carrier and prepared and/or packaged and labeled as increasing skin whitening or lightening, inhibiting, decreasing or reducing melanin production or pigmentation. These compositions may be administered topically. Examples of cosmetically acceptable carriers include, but are limited to, water, glycerin, waxes, various alcohols such as ethanol, propyl alcohol, vegetable oil, mineral oil, silicone oils, fatty esters, fatty alcohols, glycols, polyglycols or any combinations thereof. Finished compositions may be in any form suitable for topical application to the skin such as, but not limited to, aerosol spray, gel, cream, dispersion, emulsion, foam, liquid, lotion, mousse, patch, pomade, powder, pump spray, solid, solution, stick or towelette. Emulsions may include oil-in-water emulsions, water-in-oil emulsions and water-in-silicone emulsions.

The compositions may be administered as needed, daily, several times per day or in any suitable regimen such that the desired outcome is achieved. In the method, the frequency of topical applications will depend on several factors, including the desired level of inhibition of melanin production. Generally, a regimen includes application of the composition to the skin once or twice daily to include an application in the morning and/or an application in the evening. The amount of composition applied to the skin during each application may depend on several factors including level of desired results and the specific composition.

### EXAMPLE 1

### Tara Extract Demonstrated Dose Dependent Reduction of Melanin Production

To analyze the effect of the Tara extract on melanin production, an assay was performed. Aqueous extract of Tara was prepared in Dimethyl Sulfoxide (DMSO) and was tested for its capacity to reduce melanin production using Melanin inhibition assay. Extracts for the melanin inhibition analysis were prepared according the protocol described by Diwakar, et al. (Fitoterapia, 83(6), 989-95 (2012)). The Tara extract was solubilized in DMSO in concentrations of 12.5 μg/ml, 25 μg/ml and 50 μg/ml and added to freshly seeded Melan-a cells at a density of 5×10⁴ cells on a 24-well plate. Each dose of Tara extract was added to the cells in triplicate. After 48 hours, the cells were treated again with the Tara extract in DMSO and supplemented with fresh medium. The final concentration of DMSO in the culture medium was maintained at 0.05% by weight. After two additional days, melanin was extracted from the cells and quantified by normalizing with protein content, following the method described by Komatsu et al. (Pigment Cell Res. 2005, 18:447).

Melanin content from the cells treated with Tara extract was compared to melanin content from untreated cells (UT). Phenylthioruea (PTU) is well known as a tyrosinase inhibitor at 60 μg/mL and was used in the assay as a positive control and control for cell viability.

FIG. 2, is a graphical representation of some of the data collected during the assay. It can be seen that use of Tara extract resulted in a dose-dependent, or concentration-dependent, reduction in melanin production.

### EXAMPLE 2

### Tara Extract and Gotu Kola Extract Synergistically Reduce Melanin Production

To determine the effect of the combination of Tara extract and Gotu Kola on melanin production, the following assay was conducted. The test compositions listed below were solubilized in DMSO and added to Melan-a cells that were freshly seeded at 5×10⁴ cells on a 24-well plate. Each dose of the test composition was added to the cells in triplicate.

Test Compositions

1. 60 μg/ml Phenylthiourea (PTU) as a positive control for inhibiting melanin production

2. 12.5 μg/ml Gotu Kola alcohol extract

3. 12.5 μg/ml Tara extract

4. 12.5 μg/ml Gotu Kola extract plus 12.5 μg/ml Tara extract

Each test composition was added to the cells in triplicate. After 48 hours, the cells were treated again with the test composition in DMSO and supplemented with fresh medium. The final concentration of DMSO in the culture medium was maintained at 0.05% by weight. After two additional days, melanin was extracted from the cells and quantified by normalizing with protein content, following the method described by Komatsu et al. (Pigment Cell Res. 2005, 18:447).

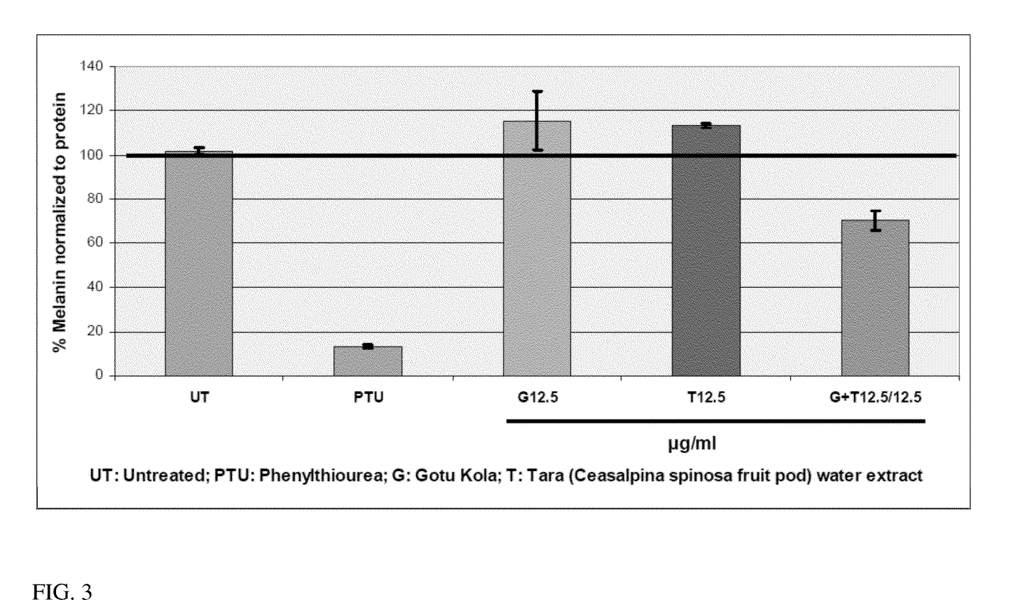

Inhibitory activity of the test compositions was reported as a percent reduction in melanin production compared to 100% melanin content from untreated cells (UT). The results are plotted in FIG. 3. It can be seen that when compared to Gotu Kola extract alone and to Tara extract alone, the combination of Tara extract and Gotu Kola extract in a substantially 1:1 ratio provided almost a 2.5 fold reduction in melanin production.

### EXAMPLE 3

### Cell Viability

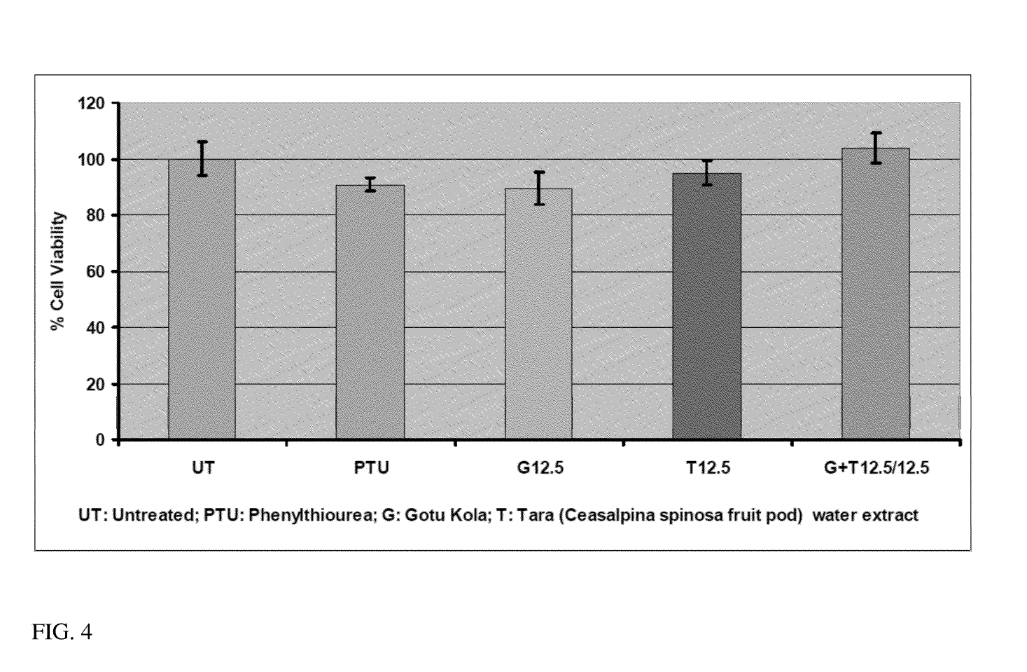

Cell viability of the test compositions prepared in Example 2 were tested by analysis with a Q-blue cell viability kit according to the instructions outlined in the manufacturer's protocol. The cell viability kit was obtained from BioAssay Systems, Hayward, Calif., Cat. No. CQBL05K FIG. 4 is a graphical representation of the resulting viability as compared to 100% viability of the untreated cells. As demonstrated by the data represented in FIG. 4, there was no significant effect on cell viability at the material concentrations tested in the assay.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

The embodiments of the invention in which an exclusive property or privilege is claimed are defined as follows:

1. A topical composition for administration to a subject, the composition comprising an extract from plants of the genus Caesalpinia in an amount effective to reduce melanin production in the subject.

2. The composition of claim 1 wherein the extract is obtained from Caesalpinia spinosa.

3. The composition of claim 2 wherein the extract is present in an amount of about 12.5 μg/ml to about 50 μg/ml in the composition.

4. The composition of claim 3 wherein the extract is present in an amount of about 12.5 μg/ml in the composition.

5. The composition of claim 3 comprising a cosmetically acceptable vehicle.

6. The composition of claim 5 wherein the cosmetically acceptable vehicle is selected from the group consisting of: water, glycerin, waxes, an alcohol, vegetable oil, mineral oil, silicone, fatty esters, fatty alcohols, glycols, polyglycols and combinations thereof.

7. The composition of claim 5 further comprising an extract of Centella asiatica.

8. The composition of claim 1 wherein the extract is obtained from a fruit pod of Caesalpinia spinosa.

9. A topical composition comprising a cosmetically acceptable vehicle, an extract of Centella asiatica, and an extract from plants of the genus Caesalpinia in an amount effective to provide a reduction in melanin production in a subject.

10. The composition of claim 9 wherein the extract is obtained from Caesalpinia spinosa.

11. The composition of claim 10 wherein the Caesalpinia spinosa extract is present in an amount of about 12.5 μg/ml to about 50 μg/ml in the composition.

12. The composition of claim 10 wherein the extract of Centella asiatica is an alcohol extract.

13. The composition of claim 12 wherein the Centella asiatica extract is present in an amount of about 12.5 μg/ml to about 50 μg/ml in the composition.

14. The composition of claim 9 wherein a concentration of the extract of Centella asiatica and a concentration of the extract from plants of the genus Caesalpinia are present in a substantially 1:1 ratio.

15. A method of whitening skin comprising topically applying to the skin a composition comprising an amount of an extract from a Caesalpinia plant extract effective to reduce melanin production, wherein the composition is in an amount and for a period of time sufficient to visibly whiten the skin.

16. The method of claim 15 wherein the composition further comprises an extract of Centella asiatica.

17. The method of claim 16 wherein the extract from the Caesalpinia plant is an extract of Caesalpinia spinosa.

18. The method of claim 17 wherein the Caesalpinia spinosa extract is present in a concentration of about 12.5 μg/ml to about 50 μg/ml.

19. The method of claim 18 wherein the Centella asiatica extract is present in a concentration of about 12.5 μg/ml to about 50 μg/ml.

20. The method of claim 15 wherein a concentration of the extract of Centella asiatica and a concentration of the extract from plants of the genus Caesalpinia are present in a substantially 1:1 ratio.
